(19) United States
(12) Patent Application Publication
(10) Pub. No.: US 2018/0140417 A1
(43) Pub. Date: May 24, 2018

(54) DEVICE FOR INSERTION INTO HUMAN OR ANIMAL BODY AND ASSOCIATED METHODS

(71) Applicant: CAMBRIDGE ENTERPRISE LIMITED, Cambridge, Cambridgeshire (GB)

(72) Inventors: Paolo De SCISCIO, Cambridge (GB); Jacob BRUBERT, Cambridge (GB)

(73) Assignee: CAMBRIDGE ENTERPRISE LIMITED, Cambridge, Cambridgeshire (GB)

(21) Appl. No.: 15/572,750

(22) PCT Filed: May 11, 2016
(86) PCT No.: PCT/GB2016/051347

§ 371 Date: Nov. 8, 2017

(30) Foreign Application Priority Data
May 13, 2015 (GB) 1508150.8

(51) Int. Cl. A61F 2/24; A61F 2/04; A61F 2/82

(52) U.S. Cl. CPC A61F 2/2418; A61F 2002/047; A61F 2/82; A61F 2/04

(57) ABSTRACT

An implant device for insertion in a human or animal body comprises a frame, wherein at least a portion of the frame is capable of reversibly transitioning between a solid state rigid mode and a liquid state flexible mode, thereby allowing the device to change its shape.

The present invention relates to devices for insertion into human or animal bodies and associated methods. Such devices include prostheses such as heart valves. In particular embodiments, the present invention relates to a prosthesis and method for transcatheter heart valve replacement using an expandable and collapsible implant.

The overall prevalence of valvular heart disease in the developed world, in particular aortic and mitral valve disease, is 2.5-3.0% with an age-dependent increase to over 13% in people >75 years (d'Arcy et al., 2011 and Kouchoukos et al., 2013). Valvular replacement may be indicated when there is narrowing of the valve, termed stenosis, or where the valve insufficiently closes resulting in backflow and/or leakage, termed regurgitation. Prosthetic heart valves comprise of an inner structure that permits uni-directional blood flow, typically one or more leaflets, attached to an outer support structure to enable anchorage to the native anatomy. Replacement with a mechanical valve comprising either a single leaflet, double leaflet, ball and cage or slit-type requires lifetime anticoagulant therapy to reduce clot formation and risk of cerebrovascular accidents. Replacement with a bioprosthetic (tissue) valve formed of either animal pericardium in the case of a xenograft or human pericardium in the case of a homograft does not require anticoagulation however has a limited durability of 15 years.

Historically, valve replacement has been performed via open-heart surgery with the native valve excised and replaced under cardiac arrest—30 day mortality rate of 2-3% in the low-risk elective setting and up to 30% in high-risk emergency patients (Walther et al., 2012 and lung et al., 2003). Procedural risks include but are not limited to cerebrovascular accident, myocardial infarction, arrhythmia, haemorrhage, wound infection and death. Surgical replacement is poorly tolerated in elderly patients. Hannan et al. (2009) reports an adjusted hazard ratio for 30-month postoperative survival of 1.57, 2.18 and 3.96 for patients aged 65-74 years, 75-84 years and >85 years, respectively. Likewise significant co-morbidities increase perioperative mortality with an odds ratio of 10.6 for emergency procedure: 4.9 for renal failure, 3.1 for end-stage heart failure and 4.3 for neurological dysfunction (Jung et al., 2005). These risks have led to the development of minimally-invasive approaches for valvular replacement that reduce perioperative mortality.

U.S. Pat. No. 6,168,614 and U.S. Pat. No. 6,582,462 to Anderson, et al., are among the first to describe a percutaneous heart valve prosthesis whereby a replacement tissue valve is mounted within an expandable metallic support structure. The first human percutaneous (or transcatheter) valve replacement was performed in 2000 by Bonhoeffer et al. in a peadiatric patient with pulmonic stenosis and insufficiency. In 2002, Cribier et al. extended the utility of transcatheter replacement to include aortic valve disease. Since that time more than 90,000 transcatheter aortic valves have been implanted in over 40 countries (Bourantas et al., 2013). Most iterations relate to a xenograft valve secured within a metallic frame. The frame is typically crimped for transcatheter delivery with deployment via balloon expansion or self expansion (in the case of shape memory alloy). Percutaneous access is achieved via the femoral artery, subclavian artery, ascending aorta or direct puncture of the left ventricle.

Initially developed to treat patients contraindicated for surgical intervention, recent years have seen a shift towards transcatheter replacement as a primary modality for patients at high-to-intermediate risk as defined by the Society of Thoracic Surgeons Predicted Risk of Mortality score. Unfortunately, implantation of current transcatheter valves is not reversible. The fixed radial configuration of the fully deployed outer support structure does not permit endovascular removal and significantly complicates surgical excision if indicated. Given a durability of 10-15 years, transcatheter valve replacement remains limited to patients aged >70 years or those who will not outlive the prosthesis. Younger patients must therefore choose between surgical replacement with a mechanical valve and subsequent anticoagulation, or a bioprosthetic valve and multiple repeat procedures. In patients with an active lifestyle, specifically those involved in contact sports or high risk employment, a bioprosthetic valve is viewed as more favourable given the lifestyle restrictions associated with management of the mechanical alternative.

Additional limitations in the prior art include the effect that crimping a rigid frame has on aspects of the valvular leaflets. Recent evidence suggests that the process of crimping a rigid frame to facilitate endovascular delivery may lead to long term structural deterioration of the leaflets (Stortecky et al., 2012 and Alavi et al., 2014 and de Buhr., 2012). Likewise, expansion of a rigid frame via balloon inflation or the shape memory effect results in imperfect matching of the prosthesis to the native geometry of the valvular annulus which results in deformation of the deployed frame. As such, long term use includes the risk of stent fracture and prosthesis migration.

Suboptimal positioning is also of considerable concern. Suboptimal positioning at time of deployment may be due to foreshortening of the prosthesis during deployment, inadequate visualization and/or limited tactile feedback. Suboptimal deployment distal to the aortic annulus is associated with increased paravalvular leak and potential obstruction of the coronary ostia. Suboptimal deployment with proximal extension into the left ventricular outflow tract may compress structures of the internal conductive system, specifically the AV node and left bundle branch necessitating a permanent pacemaker. Similarly, impingement on the mitral valve may lead to deterioration of the mitral leaflets.

A number of current devices have sought to address the problem of suboptimal deployment. In one iteration, Patent No. US 20140128963, the delivery sheath is used to recapture the prosthesis before two-thirds deployed. U.S. Pat. No. 8,246,678 describes a method of active longitudinal shortening with retraction before final implantation. U.S. Pat. No. 8,317,858 employs support clips or control arms that capture the native leaflets and align the prosthesis within the native annulus. Patent No. US 20110160846 describes an inflatable device formed in vivo for optimisation of position prior to permanent fixation. Unfortunately, the aforementioned mechanisms cannot reposition the replacement valve after full deployment. Thus the prior art does not include a heart valve prosthesis that is percutaneously deployed with the capacity to be removed after full deployment via endovascular methods.

In view of the limitations associated with the prior art, herein is disclosed the advantages of a heart valve prosthesis with capacity to transition between flexible and rigid states to facilitate implantation, repositioning and removal before and after full deployment via endovascular methods. However, such advantages are not limited to the field of heart valves, and are more generally applicable to implantable devices, as discussed in more detail herein.

In accordance with a first aspect of the present invention, there is provided an implant device for insertion in a human or animal body, the device comprising: a frame; and wherein at least a portion of the frame is capable of reversibly transitioning between a solid state rigid mode and a liquid state flexible mode, thereby allowing the device to change its shape. The ability to transition between rigid and flexible modes assists in locating, re-locating or removing the device.

The device can be or includes an implantable prosthesis such as a heart valve, cardiac stent, biliary stent or colonic stent, an inferior vena cava filter or prosthetic urinary sphincter, or an implantable catheter, surgical retractor or endoscopic speculum.

The frame can comprise elements which are not capable of reversibly transitioning between a rigid and flexible mode.

At least said portion of the frame can comprise a core surrounded by an encapsulating layer. The encapsulating layer can comprise biological or polymeric material. Said portion can be capable of reversibly transitioning between a rigid and flexible mode due to a change in the properties of the core.

Said portion of the frame can be configured to undergo the transition in phase under the influence of a stimulus. The material properties of the core are such that the core undergoes a phase transition at a temperature of from 35° C. to 80° C., more preferably from 40° C. to 70° C., and even more preferably from 45° C. to 60° C.

The encapsulation layer can comprise fluoropolymers, in particular polytetrafluoroethylene (PTFE) or expanded PTFE (ePTFE) or fluorinated ethylene propylene (FEP) or ethylene-tetrafluoroethylene (ETFE), polysiloxanes, polyurethanes, polybutylenes, polyolefins, and/or styrenic thermoplastic elastomers, in particular poly(styrene-block-isobutylene-blockstyrene) (SIBS).

Said portion or said core material can comprise a eutectic alloy, ethylene butyl acrylate copolymer, ethylene methyl acrylate copolymer, ethylene acrylic acid copolymer, ethylene methacrylic acid copolymer, polybutylene, poly(epsilon-caprolactone), thermoplastic polyolefin elastomer/plastomer, thermoplastic polyurethane elastomer, polyamide, polylactic acid, poly(n-isopropylacrylamide) and/or cellulose acetate butyrate.

Said portion, or said core if the portion comprises an encapsulating layer, can have a Young's modulus of at least 0.1 GPa in the rigid mode, preferably at least 1 GPa. Said portion, or said core if the portion comprises an encapsulating layer, can have a Young's modulus of less than 0.1 GPa in the flexible mode, preferably less than 0.01 GPa.

According to another aspect of the invention, there is provided a method of using the device of the previous aspect, the method comprising: causing the device to undergo transition from one of the rigid or flexible modes to the other.

According to another aspect of the invention, there is provided a method of inserting the implant device of the first aspect into a human or animal body, the method comprising: causing the device to undergo transition from the rigid mode to the flexible mode; shaping the device into a deployed configuration; and causing the device to undergo transition from the flexible mode to the rigid mode. The method can further comprise inserting and implanting the device into the human or animal body either before or after the step of causing the device to undergo transition from the rigid mode to the flexible mode.

According to another aspect of the invention, there is provided a method of moving, reshaping, or removing from a human or animal body, the device of the first aspect, the method comprising: causing the device to undergo transition from the rigid mode to the flexible mode; and moving the device, optionally including reshaping or removing the device from the human or animal body.

The step of causing the device to undergo transition from the rigid mode to the flexible mode in any of the method aspects can comprise heating said portion of the device. The source of heat can be external to the body, internal to the body but separated from the frame, or in direct contact with the frame.

An advantage of the present invention is the provision of a frame that can be returned to its flexible state, after full deployment, to enable removal via endovascular methods in particular applications. That is, the invention can provide a transcatheter heart valve that is endovascularly removable even after it has been fully deployed. Similarly, the invention can provide an inferior vena cava filter that is endovascularly removable after full deployment or an oesophageal stent that is endoscopically removable after full deployment.

Another advantage of the present invention is the provision of a frame adaptable to the native geometry. That is, there is provided a frame; and at least a portion of the frame is capable of reversibly transitioning between a rigid mode and a flexible mode, thereby allowing the device to change its shape. Such a frame can have applications in other fields besides cardiovascular medical devices. That is, the invention can provide a colonic stent or bariatric sleeve that conform to the native geometry. Similarly, the invention can provide an implantable urinary sphincter that adapts its geometry to regulate urinary flow.

Still another advantage of the present invention is the provision of a heart valve prosthesis with a flexible profile thus reduced stresses on the leaflets during endovascular delivery.

The invention is described below with reference to exemplary embodiments and the accompanying drawings, in which:

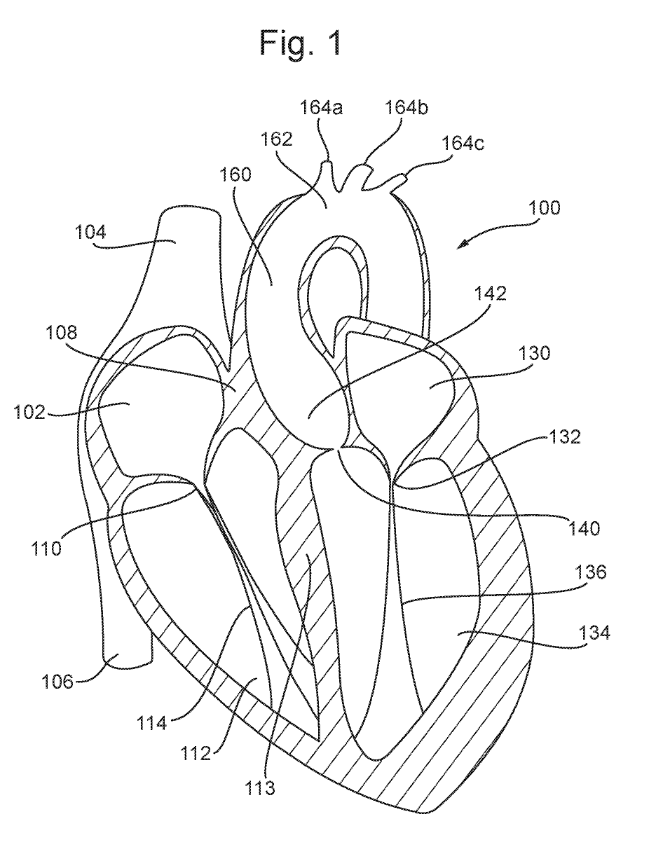

FIG. 1 is a schematic coronal view of the heart including valvular structures and major cardiac vessels.

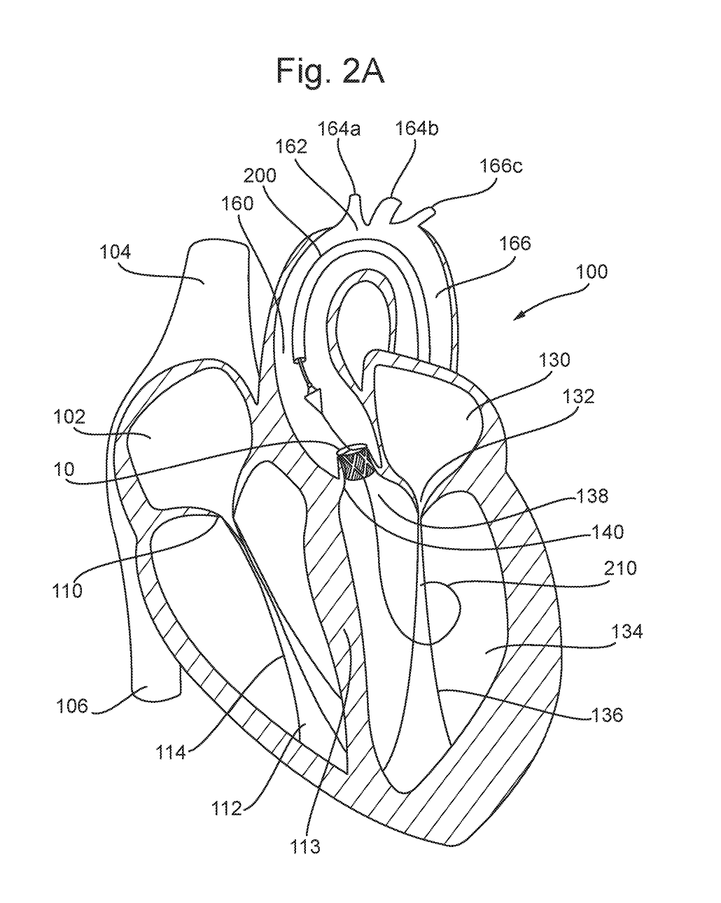

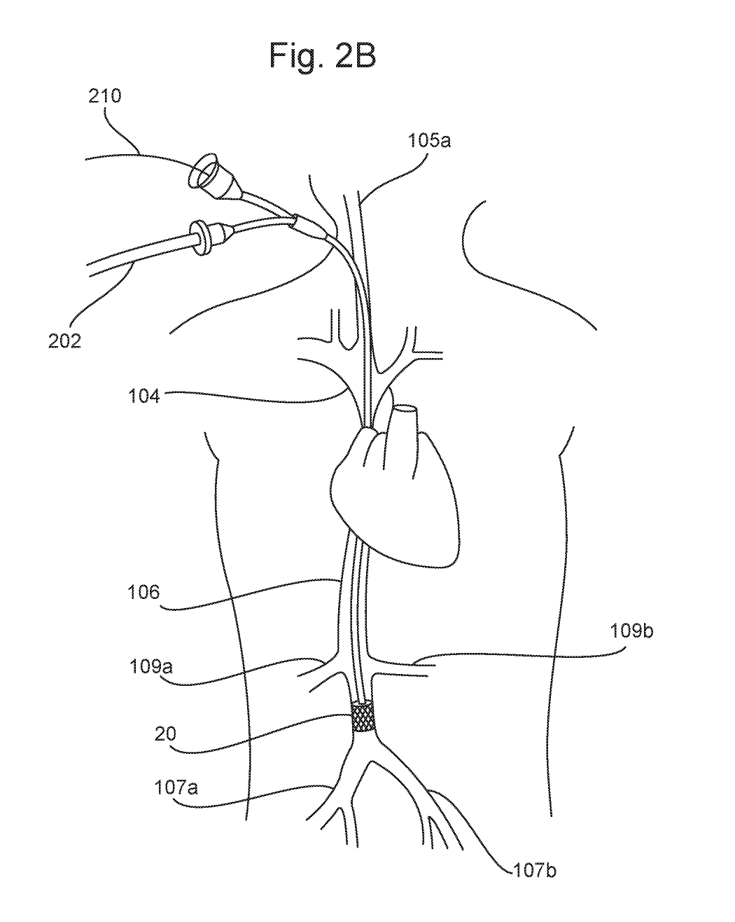

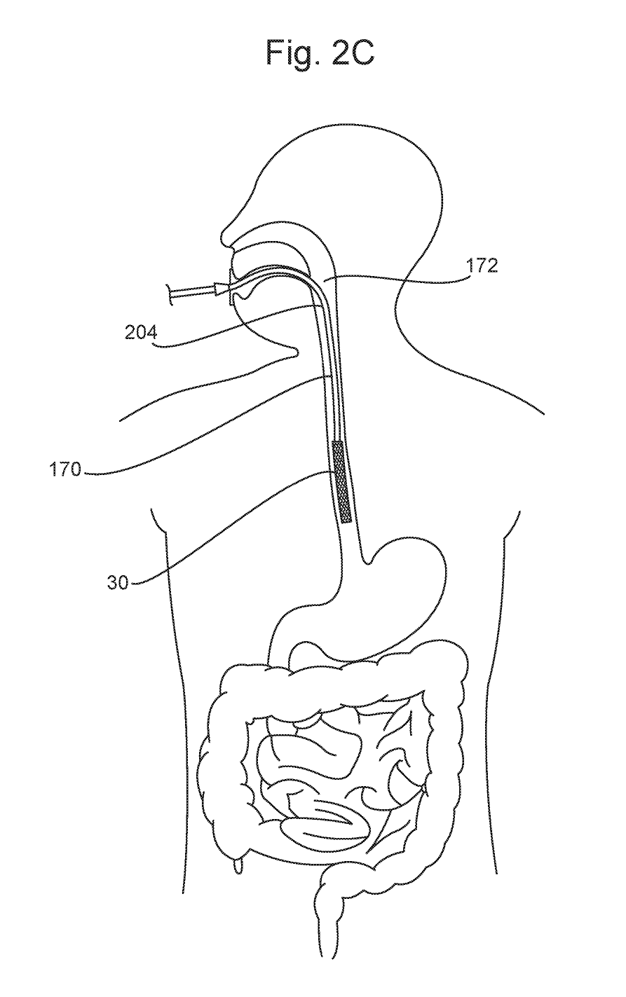

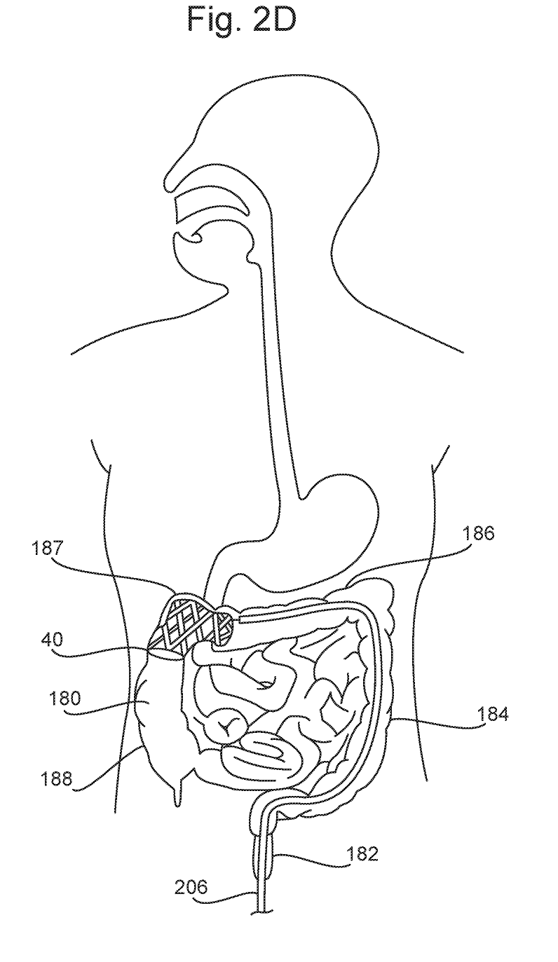

FIG. 2A is a schematic coronal view of the heart and vessels demonstrating deployment of a prosthetic aortic valve according to one embodiment of the present invention.

FIG. 2B is a schematic coronal view of the heart and vessels demonstrating deployment of an inferior vena cava filter according to a further embodiment of the present invention.

FIG. 2C is a schematic coronal view of the oesophagus and adjacent structures demonstrating deployment of an oesophageal stent according to a further embodiment of the present invention.

FIG. 2D is a schematic coronal view of the colon and adjacent structures demonstrating deployment of a colonic stent according to a further embodiment of the present invention.

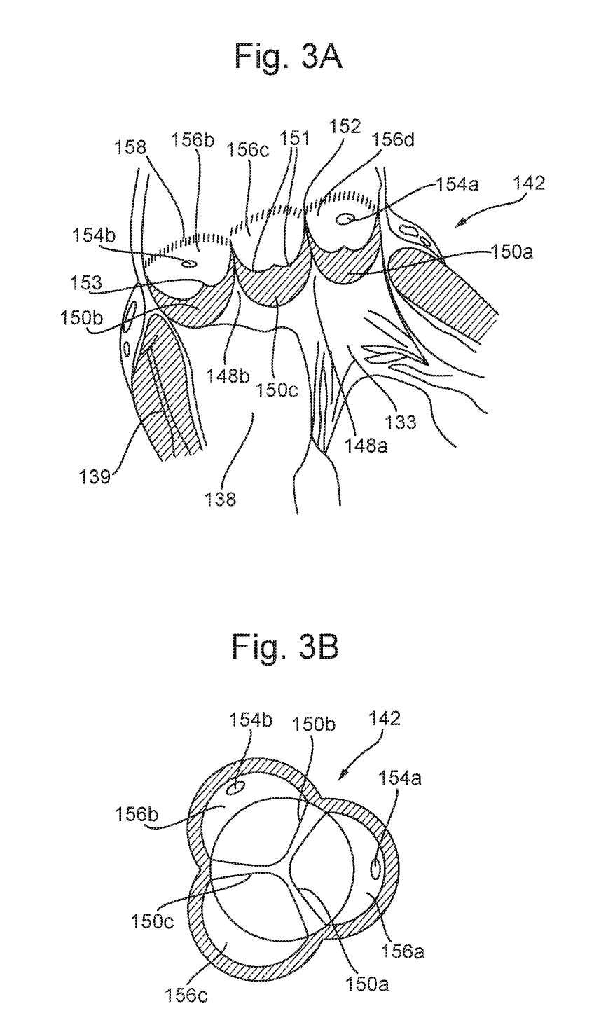

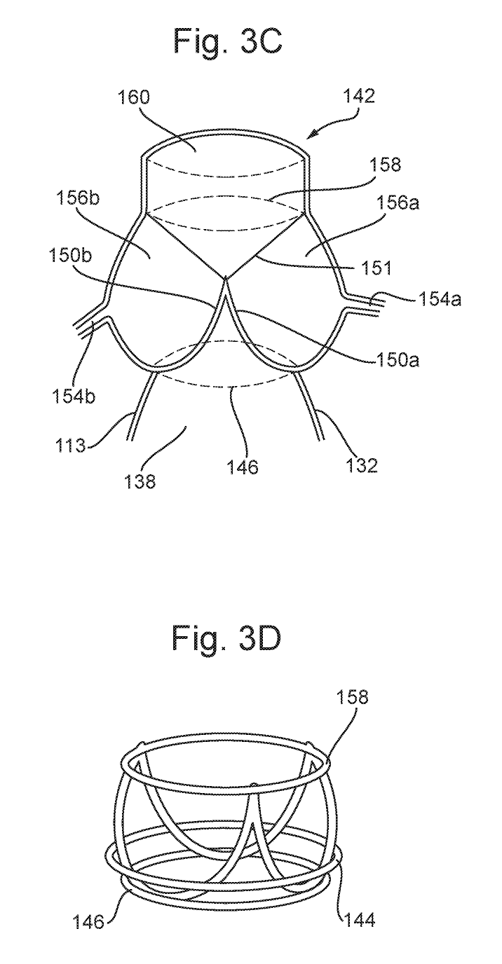

FIGS. 3A-3D are schematic isometric view of the native aortic root as a site for prosthetic valve replacement according to one embodiment of the present invention.

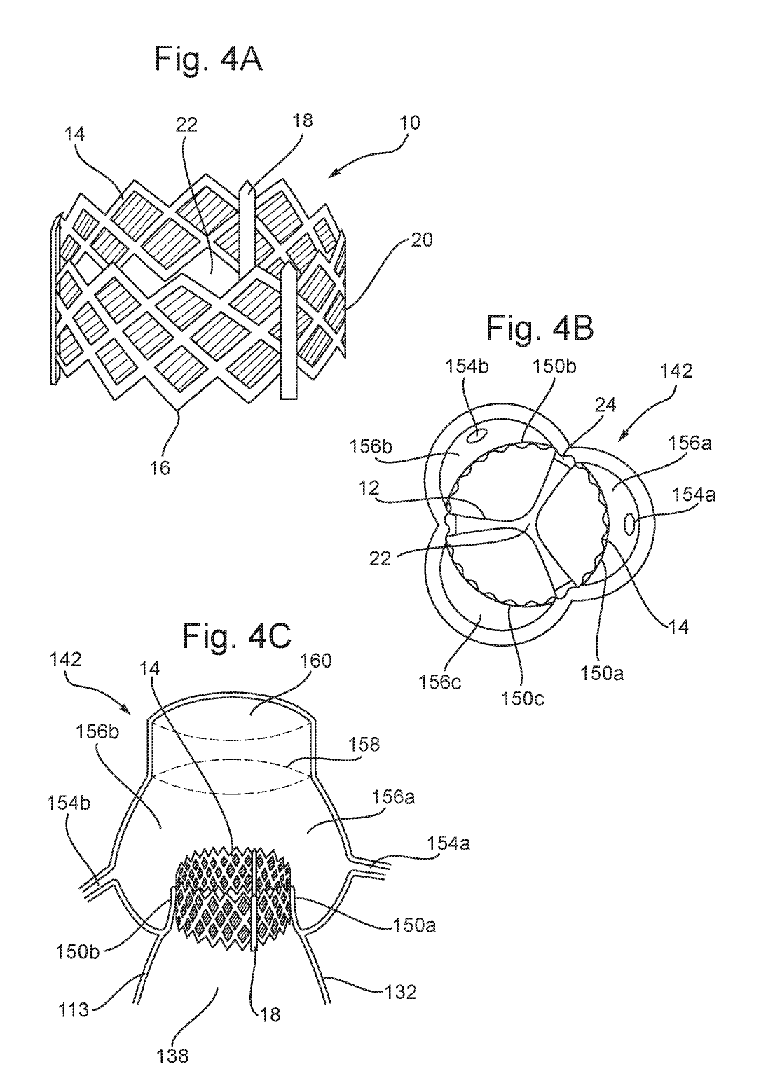

FIGS. 4A-4C are schematic isometric views detailing the prosthesis of FIG. 2, positioned within the native aortic valve of FIG. 3A.

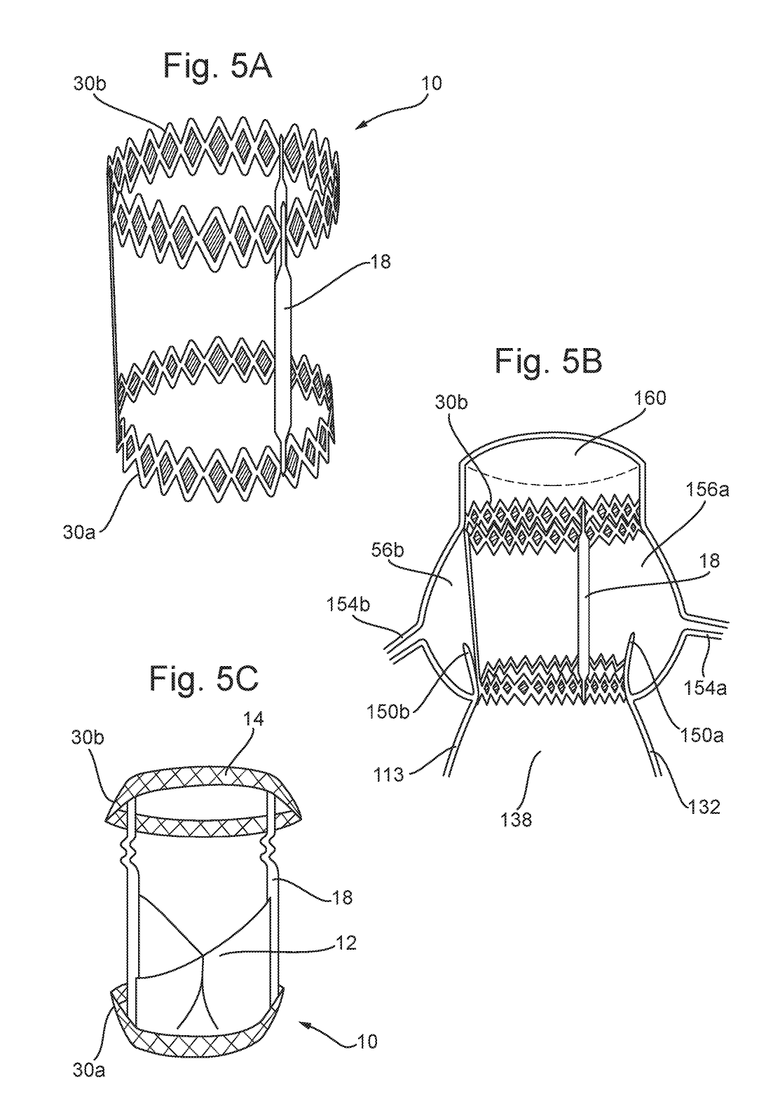

FIGS. 5A-5C are schematic isometric views detailing two modified embodiments of an implant positioned within the native aortic valve.

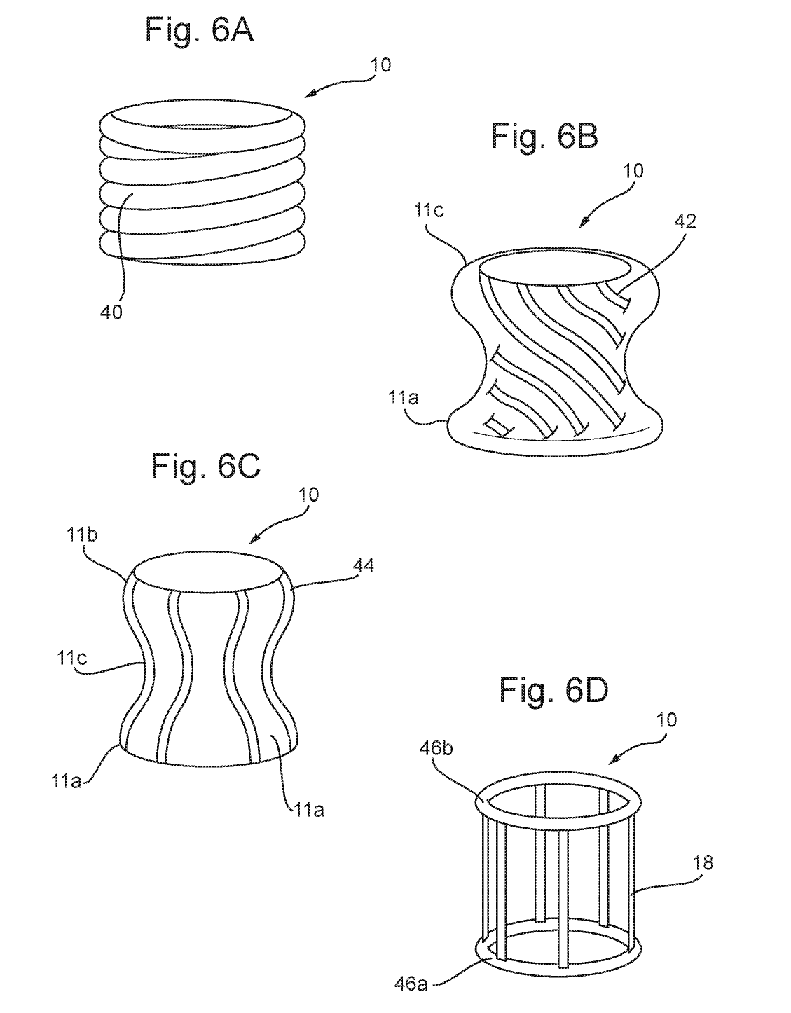

FIGS. 6A-6D are schematic isometric views detailing four other embodiments of an implant positioned within the native aortic valve.

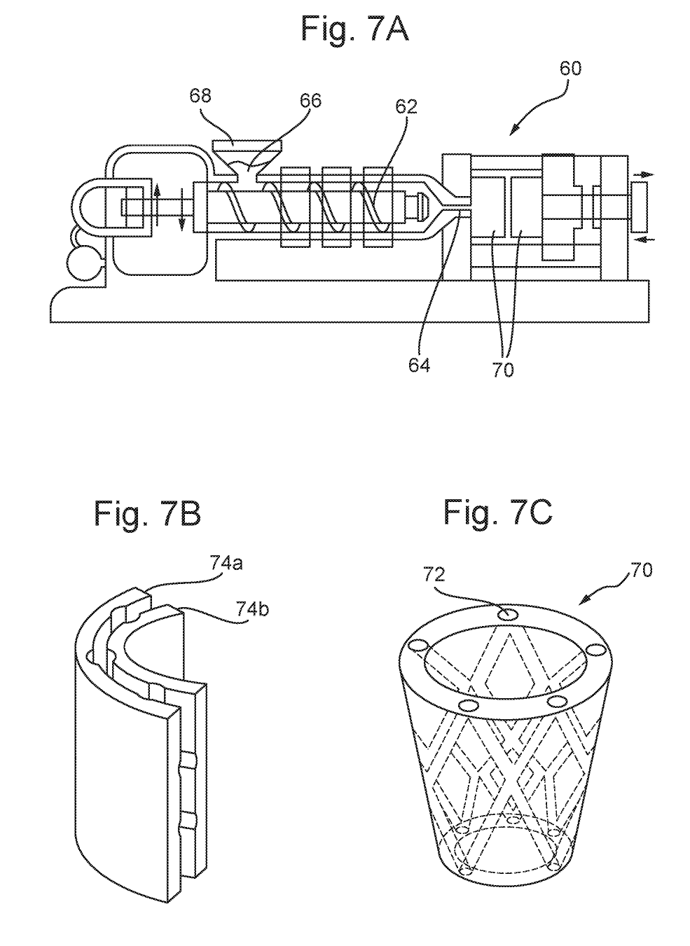

FIGS. 7A-7C are schematic cross-sectional views of an apparatus and method for fabrication of the frame of one embodiment of the proposed invention.

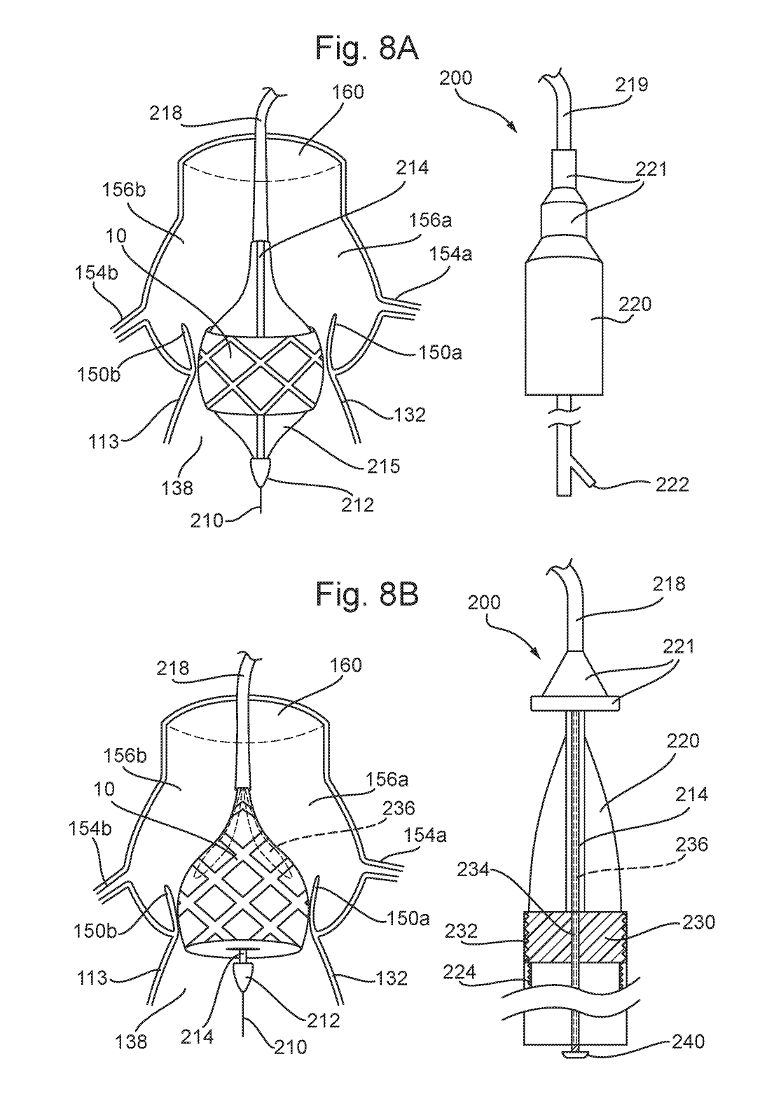

FIGS. 8A-8B are schematic isometric views of a delivery apparatus for deployment of the prosthesis of FIG. 2, positioned within the native aortic valve of FIG. 3A.

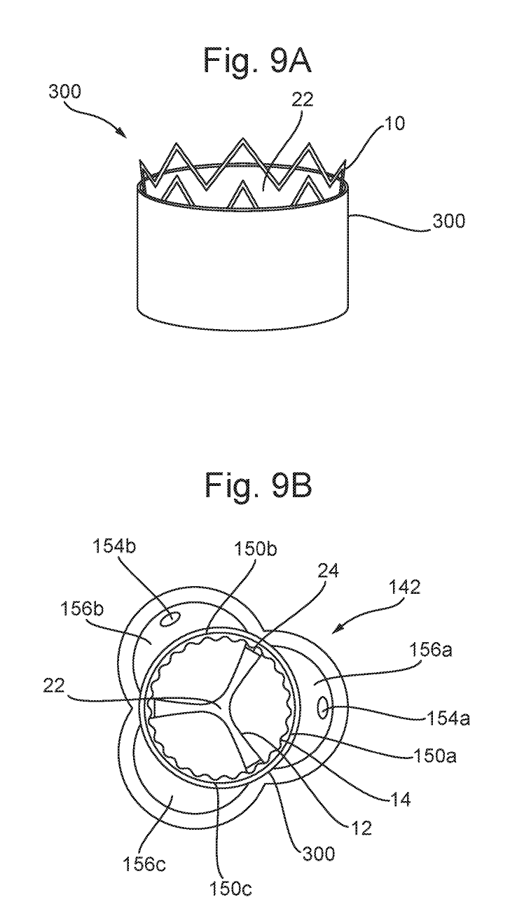

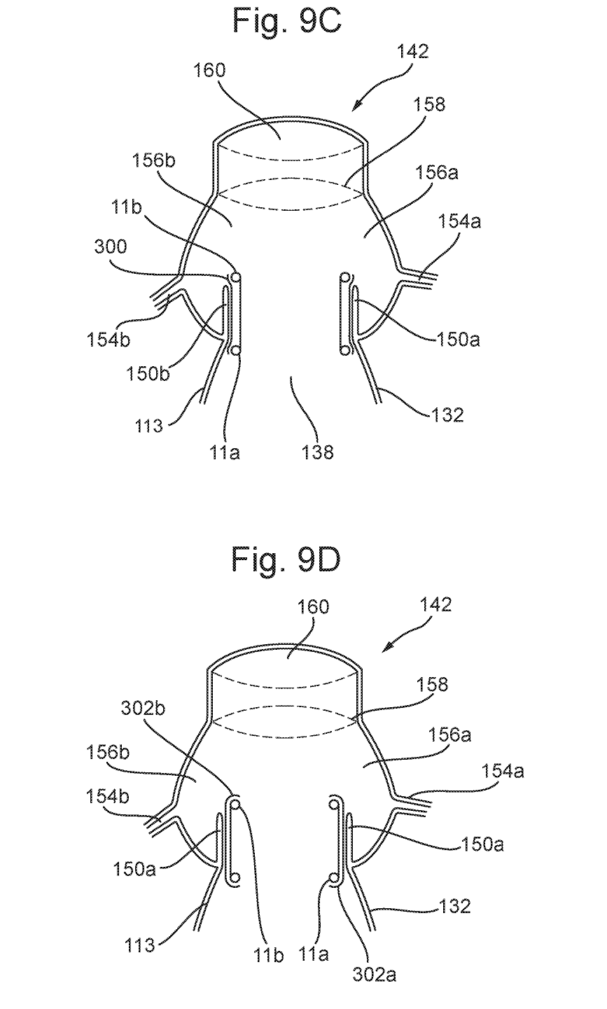

FIGS. 9A-9D are schematic isometric views of a method for establishing an outer support structure within the native aortic valve wherein a prosthesis is deployed within said structure.

The present invention relates to devices for insertion and preferably implantation in to a human or animal body, in particular prostheses or catheters, and methods for using such devices. The illustrative examples serve to describe preferred embodiments of the present invention without limitation of said invention.

Although the description below primarily focuses, for consistency and clarity, on the application of the invention in the context of the endovascular placement of an aortic heart valve, the invention may also be applied to other devices designed for insertion into a human or animal body. Such insertion may be short-term or long-term. The skilled reader will appreciate how the teaching of the present document may be applied in these other scenarios. For example, other devices in which the invention may be applied include (but are not limited to):
other valve replacements (e.g. mitral, pulmonic, tricuspid valves);
vascular stents, particularly for larger vessels, such as the abdominal aorta and illiac arteries for example;
non-cardiac stents, such as oesophageal or colonic stents for example;
inferior vena cava filters;
bariatric sleeves or bands;
prosthetic urinary sphincters;
otorhinolaryngology implants such as tympanostomy tubes;
catheters, preferably implantable, that would benefit from having a flexible and rigid state, such as indwelling sensors, basket catheters and expandable catheters;
surgical retractors; and
endoscopic specula.

In each of these examples, the device for insertion and preferably implantation into the body benefits from the ability to transition between a flexible and rigid state, either to facilitate insertion before a final placement of the device, or to facilitate repositioning of a device that was incorrectly placed (or has moved subsequent to placement) or to facilitate the eventual removal of the device, for example. Although the specifics of how such functionality might be exploited will differ from device to device, the underlying benefit the invention provides is the same and is readily appreciated by those skilled in the art.

It is further noted that the disclosure below includes discussion of devices undergoing reversible transitions between flexible and rigid states. In this context, ‘flexible’ is intended to mean that the device (or at least parts of the device) can be reshaped in a way that allows the device to conform to its surroundings with little resistance. Preferably, this does not require any material ‘yielding’ e.g. because the flexibility is achieved through part of the device becoming liquid (preferably contained within an encapsulating material, as discussed below, and of course the encapsulation itself will provide some small resistance to deformation). While in the liquid phase, said portion of the device is soft and therefore malleable by way of catheter manipulation, for example. In so doing, said portion of the device may take the geometry of adjacent structures on contact with said structures. If the device or device part remains solid after the transition to the flexible state, or if the part is a liquid enclosed in encapsulation, it preferably exhibits a Young's modulus of less than 1 GPa, preferably less than 0.1 GPa. However, it will be apparent that what qualifies as flexible will depend on the particular application in question—in some scenarios a particular Young's modulus corresponding to ‘flexible’ state may be the normal ‘rigid’ modulus of a device used for another application. The skilled person will understand that in a ‘flexible’ mode the device is usually unsuitable for maintaining its position in the body it is being inserted into.

‘Rigid’ is intended to mean that the device (or the relevant parts of the device) can maintain its overall shape and structure. In this context, a ‘rigid’ part of the device may be able to bend (e.g. if it is made of metal) but will resist plastic deformation. In some contexts, rigid can mean that the device or part of the device preferably has a Young's modulus of at least 0.1 GPa, preferably at least 1 GPa. However, as mentioned above, it will be apparent that what qualifies as rigid will depend on the particular application in question—in some scenarios a particular Young's modulus corresponding to a ‘rigid’ state may be the normal ‘flexible’ modulus of a device used for another application. The skilled person will understand that in a ‘rigid’ mode, provided the device has been appropriately shaped, the device is usually able to maintain its position in the body it has been inserted into.

In general, for any particular application, the invention relates to change in the rigidity/flexibility of the prosthesis by way of a portion of the device, preferably the encapsulated material, transitioning from a solid phase to a liquid phase or vice versa. As such it is preferred that the change in Young's modulus between the flexible and rigid states is at least a factor of 10. That is, the ratio of the rigid modulus to the flexible modulus (E_{rigid}/E_{flexible}) is 10 or more, preferably 100 or more.

Finally, ‘reversible’ is intended to mean not only that the transition can be ‘undone’ (i.e. to allow the device or device part to transition at least from the rigid state, to the reversible state and back again to the rigid state), but also to mean that such transitions occur without detriment to the overall device. That is, the device may change in shape (that being a benefit imparted by the ability to undergo such transitions), but the transitions occur in a manner which allow the device to maintain its overall structural integrity (i.e. even if the overall shape of the structure changes).

In an exemplary embodiment, the device is a medical prosthesis, particularly a device comprising a replacement aortic heart valve. The frame of the device contains a prosthetic valve for replacement of the aortic valve. The frame can comprise a material capable of a transition between a rigid and a flexible mode. Such a transition can be achieved, for example, using a eutectic alloy that can undergo a reversible solid-to-liquid phase transition at a bio-compatible temperature. The alloy can be encapsulated by a second material compatible with biological tissue. Such a scenario is discussed in detail below with reference to the figures. The replacement valve has been omitted in all but FIGS. 2, 4B, 5C and 10B to assist in illustrating the support structure of the present invention. In the following descriptions, ‘proximal’ and ‘distal’ are referenced from the perspective of the heart unless otherwise specified such that the proximal direction is that closest to the heart.

FIG. 1 is a schematic coronal view of the human heart 100 including valvular structures and major cardiac vessels. A detailed view of the interior of the heart provides an important reference for percutaneous intervention with access from either the venous system or arterial circulation. Using the flow of blood as a reference, three vessels carrying deoxygenated venous blood drain into the right atrium 102 namely the superior vena cava 104, inferior vena cava 106 and coronary sinus. The atrial septum 108 forms the postero-medial wall separating the right 102 and left atrium 130. The tricuspid valve 110, typically comprised of three leaflets, is located in the floor of the right atrium 102 opening into the base of the right ventricle 112. The right ventricle 112, separated from the left ventricle 134 by the ventricular septum 113, contains three papillary muscles that support the three valvular leaflets via thin chordae tendineae projections 114. These muscles maintain closure of the tricuspid valve 110 during ventricular systole to ensure forward flow of blood through the tri-leaflet pulmonic valve (not shown) and into the pulmonary circulation.

Oxygenated blood re-enters the heart via drainage of the four pulmonary veins into the posterior aspect of the left atrium 130. The bicuspid mitral valve 132 opens into the base of the left ventricle 134 with the inlet and outlet portions of the chamber separated by the anterior leaflet. Two larger papillary muscles attach via thicker chordae tendineae 136 to the leaflets of the mitral valve 132. The aortic root 142 arises from the base of the left ventricle 134, extending as the ascending aorta 160 and the aortic arch 162 recognised by its branches 164a, 164b, 164c. Systolic contraction of the left ventricle 134 results in forward flow of blood through the aortic valve 140, typically comprised of three leaflets.

FIG. 2A details endovascular deployment of a heart valve prosthesis 10, which is one embodiment of a device for insertion and implantation into a human or animal body according to the present invention. The prosthesis 10 is deployed within the native aortic valve 140. During deployment, a delivery apparatus 200 having the prosthesis 10 encapsulated therein can be advanced via retrograde approach over a previously positioned guide wire 210. Preferably, the delivery apparatus 200 is advanced through the descending aorta 166, aortic arch 162 and ascending aorta 160 to the site of implantation, as described by Andersen et al., in U.S. Pat. No. 6,168,614 which is hereby incorporated by reference herein. Alternative access can be established via the subclavian artery 164c, ascending aorta 160 or axillary artery. Under fluoroscopic guidance, for example, the delivery apparatus 200 can be positioned such that the proximal end of the prosthesis 10, when deployed, sits within the aortic annulus 144 with minimal extension into the left ventricular outflow tract 138.

FIG. 2B details endovascular deployment of an inferior vena cava filter 20, as another embodiment of the present invention. The filter 20 is deployed within the inferior vena cave 106. During deployment, a delivery apparatus 202 having the filter 20 encapsulated therein may be advanced over a previously positioned guide wire 210. Preferably, the delivery apparatus 202 is advanced through the right 105a or left internal jugular vein to the site of implantation, as described by Palmaz, in U.S. Pat. No. 4,793,348 which is hereby incorporated by reference herein. Alternative access can be established via the right or left common femoral vein 107a, 107b. Under fluoroscopic guidance, for example, the delivery apparatus 202 can be positioned such that the proximal end of the filter 20, when deployed, sits within the inferior vena cava 106 distal to the renal veins 109a, 109b.

FIG. 2C details endoscopic deployment of an oesophageal stent 30 as yet another embodiment of the present invention. The stent 30 is deployed within the oesophageal lumen 170. During deployment, a delivery apparatus 204 having the stent 30 encapsulated therein may be advanced through the laryngopharynx 172 to the site of implantation. Preferably, the proximal end of the stent 30, when deployed, sits within the oesophagus superior to the lower oesophageal sphincter.

FIG. 2D details endoscopic deployment of a colonic stent 40 as yet another embodiment of the present invention. Said stent 40 is deployed within the colonic lumen 180 at the hepatic flexure 187. During deployment, a delivery apparatus 206 having the stent 40 encapsulated therein may be advanced through the rectum 182, descending colon 184 and transverse colon 186 to the site of implantation. Preferably, the proximal end of the stent 40, when deployed, extends from the ascending colon 188, across the hepatic flexure 185, and into the transverse colon 186.

It is clear from the illustrations in FIGS. 2A-2D that the principles of the invention may be used in various implantable devices. The skilled person will readily understand the pertinent features that are applicable to the invention in general, and will be able to apply them to different types of devices. As such, herein, for simplicity of example, the present invention is described in the context of aortic valve replacement whereby the prosthesis 10 is deployed to function within the native aortic valve. Said prosthesis 10 or some other embodiment may similarly be used to replace the tricuspid, pulmonic or mitral valve.

FIGS. 3A-3D illustrates further detail of the anatomy surrounding the native aortic valve 140 as an implantation site for one embodiment of the present invention. With reference to FIG. 3A, delivery and optimal positioning within the aortic root 142 is dependent on the left and right coronary ostium superiorly 154a, 154b, internal conductive system medio-inferiorly 139 and the anterior leaflet of the mitral valve latero-posteriorly 133. Inclusive in the anatomical complex of the aortic root 142 is the left ventricular outflow tract 138, aortic annulus 144, leaflets 150, inter-leaflet trigones 148a, 148b, sinuses of Valsalva 156a, 156b, 156c, sinotubular junction 158 and ascending aorta 160. The native leaflets 150 are semilunar in appearance supported in a crown-like structure. The 3 leaflets are named in accordance with the underlying sinuses of Valsalva and coronary ostia—left 150a, right 150b and non-coronary 150c. Each cusp possesses 2 free margins 151 with central nodules of Arantius 153 representing the point of contact on cusp closure. The point of insertion of adjacent cusps into the aortic wall marks the valvular commissures 152. Of note, the membranous trigone 148b bounded by the right 150b and non-coronary leaflets 150c faces the internal conduction system traversing the ventricular septum 139. Deployment of a replacement prosthesis 10 as in FIG. 2 may, if over-expanded or over-extended within the outflow tract 138 by greater than 2-3 mm, lead to conduction abnormality, permanent pacemaker implantation and/or impaired mitral valve function. Such complications necessitate careful consideration of the device size and how this impacts placement, function and risk of adverse events.

FIGS. 3B-3D show further axial and coronal views of the aortic root 142. The aortic annulus 144 represents the junction between the myocardium of the left ventricular outflow tract 138 and the fibroelastic tissue of the aorta. The bases (or hinges) of the leaflets form what is known as the ‘virtual’ aortic annulus 146—a circular ring at the level of the nadir of the leaflet attachments. The point of smallest diameter within the aortic orifice largely determines prosthesis size and position. Inequalities in native leaflet 150 size or thickness can potentially distort estimation of the aortic annulus 144 leading to inaccurate sizing of the prosthesis 10. Over-sizing can create folds from excess or redundant leaflet tissue reducing valvular performance and durability. In contrast, under-sizing effectively establishes a stenotic left ventricular outflow tract 138 perpetuating the sequelae of aortic stenosis. Additionally, the location of the left 154a and right 154b coronary arteries within the two anterior sinuses of Valsalva 156a, 156b immediately below the sinutobular junction 158 necessitate design consideration.

In the embodiments described herein, the prosthesis 10 of FIG. 2 comprises a tubular support structure or frame which may be anchored within the native valvular annulus, and a replacement valve secured within its lumen to control blood flow. The prosthesis 10 may take a number of geometric forms to achieve anchorage within the native valvular annulus. Regardless of the geometric form, preferred embodiments of the frame of the prosthesis 10 (and of the device of the invention more widely) comprise encapsulated material capable of transition between a flexible and a rigid state (for example a phase transition between liquid and solid states). This encapsulation of a core allows the frame to shift between a flexible and rigid state under the influence of a stimulus which can cause the core to lose its structure integrity (e.g. because it becomes liquid) without the overall device losing its structural integrity. The aforementioned ‘stimulus’ is any suitable stimulus that can be applied from outside the device (and optionally from outside the body in which the device is being inserted), to initiate the change in flexibility/rigidity. The active application of a stimulus may be required in some cases to initiate the change in mode in both directions. In other applications, the stimulus for (e.g.) initiating the return to the rigid mode could be the cessation of a continuous stimulus required to maintain the flexible mode.

Such stimuli could include the provision or removal of heat, or an electrical charge, or any other form of energy suitable for bringing about a change (such as a change in state) of a given material. Preferably, when considering transitions (e.g. such as phase transitions) brought about by a change in temperature, said material should possess a transition temperature within a range of from 35° C. to 80° C., more preferably from 40° C. to 70° C., more preferably from 45° C. to 60° C. This allows for a stimulus that can cause the prosthesis 10 to achieve a state in which the frame is flexible for delivery, positioning, repositioning or removal and rigid for fixation, whilst avoiding damage to surrounding tissue through overheating.

Preferably, if comprising more than one element, said material exists as a eutectic system such that all its components transition at a specific (eutectic) temperature.

In some embodiments, it may be desirable to use resistive heating techniques to induce a phase shift brought about by a change in temperature. In that case, said material should exhibit adequate electrical conductivity. In some cases the energy required to trigger transition may be transferred by radiation, with a source that may be external to the body, or internal to the body and separated from, or in direct contact with, the frame. In another embodiment the energy transfer for transition may be conductive in which case the source is directly connected to the frame.

The specific design or mechanism for heat activation of a phase transition can be optimised to enable efficient and accurate delivery and may include direct current or electromagnetic induction. U.S. Pat. No. 8,382,834 to Prescott and WO 1,997,022,290 to Guenther and Schmitz-Rode describe methods of inductively heating shape memory alloy within the human body using coils positioned either within or external to an implant.

By way of example and not limitation, the encapsulated material may comprise one or more metal elements such as: Bismuth, Indium, Zinc, Tin, Aluminium, Gallium, Silver, Gold, Copper, Palladium, Beryllium, Nickel, Titanium, Antimony. Similarly, combinations of different elements not limited to metals may be used to achieve the desired physical properties, in particular a transition condition (such as a transition temperature) compatible with biological tissue. Preferably, the encapsulation does not undergo a transition upon the influence of the external stimulus that causes a rigid/flexible transition in the core material.

It may be advantageous to select a radio-opaque material to enhance visualisation of the prosthesis under fluoroscopic guidance. Markers may instead be used such as gold, platinum or other appropriate materials. Additionally, it may be advantageous to select a material in which each of its components is biocompatible as a means to manage toxicity risk in the case of leaching into the vasculature.

One example of a eutectic alloy compatible with the disclosed application is described by Moelans N, Hari Kumar K C, Wollants P, 2003. Thermodynamic optimization of the lead-free solder system Bi—In—Sn—Zn, Journal of Alloys and Compounds, 360(1-2): 98-106. The alloy comprises:

(1) Bismuth, preferably having a purity of at least 99.99 mol %, in a proportion from 15.0 to 60.0 weight percent, preferably from 30.0 to 45.0 weight percent, more preferably 35.0 weight percent,

(2) Indium, preferably having a purity of at least 99.99 mol %, in a proportion from 30.0 to 70.0 weight percent, preferably from 45.0 to 55.0 weight percent, more preferably 48.6 weight percent,

(3) Tin, preferably having a purity of at least 99.99 mol %, in a proportion from 0.0 to 30.0 weight percent, preferably from 10.0 to 20.0 weight percent, more preferably 16.0 weight percent, and

(4) Zinc, preferably having a purity of at least 99.99 mol %, in a proportion from 0.0 to 5.0 weight percent, preferably 0.4 weight percent.

In the most preferable embodiment as set out above, this results in a material demonstrating a transition temperature, modulus of elasticity, compressive strength and bending modulus of 57.3° C., 9 GPa, 38 MPa and 6.4 GPa, respectively.

Variations of the aforementioned components or the addition of elements including Silver, Gold, Aluminium, Nickel and/or Gallium may be preferred to achieve the desired thermal, mechanical and/or electrical properties.

Alternatively, it may be advantageous in terms of biocompatibility, manufacture and/or clinical function to use one or more polymers, for example Ethylene butyl acrylate copolymer, Ethylene methyl acrylate copolymer, Ethylene acrylic acid copolymer, Ethylene methacrylic acid copolymer, Polybutylene, Poly(epsilon-caprolacton), Thermoplastic polyolefin elastomer/plastomer, Thermoplastic polyurethane elastomer, Polyamide, Polylactic acid, Poly(n-isopropylacrylamide) and/or Cellulose acetate butyrate.

Tables 1 and 2 provide examples of materials which could be used to provide a suitable temperature triggered transition in rigidity/flexibility.

TABLE 1
Materials exhibiting a melting point for providing a rigid/flexible transition:

| | | Melting | Young's | |
|---|---|---|---|---|
| | | point | Modulus | Mechanism |
| Material | Type | (C.) | (GPa) | of Transition |
| Bi (35.0), In (48.6), Sn (16.0), | Eutectic Alloy | 57.3 | 8.5-9.5 | Temperature |
| Zn (0.4) | | | | |
| Bi (32.5), In (51.0), Sn (16.5) | Eutectic Alloy | 60.5 | 8.5-9.5 | Temperature |
| Bi (33.7), In (66.3) | Eutectic Alloy | 72 | — | Temperature |
| Bi (57.0), In (26.0), Sn (17.0) | Eutectic Alloy | 79 | — | Temperature |
| Bi (54.0), In (29.7), Sn (16.3) | Eutectic Alloy | 81 | — | Temperature |
| Gallium | Metal | 30 | 9.8 | Temperature |
| Rubidium | Metal | 39 | 2 | Temperature |
| Thermoplastic Polyolefin | Polymer | 51-77 | 0.01-0.035 | Temperature |
| Elastomer/Plastomer | | | | |
| Poly(epsilon-caprolacton) | Polymer | 53-62 | 0.4 | Temperature |
| Thermoplastic Polyurethane | Polymer | 78-118 | 0.03 | Temperature |
| Elastomer | | | | |
| Ethylene acrylic acid | Polymer | 81.9-93.9 | 0.1-0.38 | Temperature |
| copolymer (EAA) or | | | | |
| ethylene methacrylic acid | | | | |
| copolymer (EMAA) | | | | |
| (partial sodium or zinc salt) | | | | |
| Polybutylene | Polymer | 83-97 | 0.069-0.103 | Temperature |
| Ethylene butyl acrylate | Polymer | 86-102 | 0.025-0.04 | Temperature |
| copolymer | | | | |
| Ethylene methyl acrylate | Polymer | 86-102 | 0.025-0.04 | Temperature |
| copolymer | | | | |

TABLE 2
Materials exhibiting a glass transition for providing a rigid/flexible transition:

| | | Glass | Young's | |
|---|---|---|---|---|
| | | transition | Modulus | Mechanism |
| Material | Type | (C.) | (GPa) | of Transition |
| Polyamide | Polymer | 36-48 | 1.06-1.33 | Temperature |
| Cellulose acetate butyrate | Polymer | 51-60 | 0.9-0.9 | Temperature |
| PLA (30% natural fibre) | Polymer | 52-54 | 5.19-5.32 | Temperature |
| Coordination polymer | Polymer | 90 | Crystalline | Temperature |
| crystals with liquid solid | | | | |
| transition, for example | | | | |
| [Zn3(H2PO4)6(H2O)3]•H(2- | | | | |
| MeBim) | | | | |

The material undergoing the transition may be encapsulated by one or more materials. In some cases, this may not be required if the transition is of a type that allows the material to maintain a 3D structure whilst also becoming flexible. In other cases, such as for a liquid phase transition, the encapsulation ensures that the material remains contained within the 3D structure of the prosthesis 10 whilst in the flexible state.

The encapsulating materials, which may be a composite, preferably consists of a material which is impermeable such that leaching from the material undergoing the transition (e.g. of metal ions) is limited. The encapsulating material or coating in contact with tissue is also preferably compatible with biological tissue. When dealing with a thermally induced transition, it is preferable that the encapsulating material(s) should be capable of limiting heat transfer to the surrounding tissue.

Example encapsulating materials may include fluoropolymers (e.g. polytetrafluoroethylene (PTFE), expanded PTFE (ePTFE), fluorinated ethylene propylene (FEP), ethylene-tetrafluoroethylene (ETFE)), polysiloxanes, polyurethanes, polybutylenes, styrenic thermoplastic elastomers (e.g. poly(styrene-block-isobutylene-blockstyrene) (SIBS)), or other clinically implantable material.

The encapsulation may be manufactured via casting, extruding, sintering, moulding, thermoforming or other generally known methods. It may also be desirable to apply a biological or chemical coating or fixation treatments to the encapsulation to improve compatibility with surrounding tissue. Example coating materials may include silver, zwitterionic polymer, or other functionalised poly(ethylene glycol) coatings. Said coatings may be applied to the encapsulating layer using methods such as vapour deposition, sputtering, or other methods known to those skilled in the art.

Referring now to FIG. 4A, one embodiment of the prosthesis 10 comprises a frame 14. In the depicted example, the frame 14 is in the form of a diamond cell lattice. The lattice members of the frame 14 are fabricated from the encapsulated alloy. The frame 10 can contain a replacement valve, comprising of biological tissue, polymer, pure metal, alloy or a combination of the aforementioned. In some embodiments, the valve comprises between 1 and 4 leaflets, more preferably 2 or 3 leaflets.

It should be noted that in some embodiments, the prosthesis 10 can comprise an additional or plurality of outer supports to be implanted concurrently. Said supports are outer encapsulating structures whereby the inner prosthesis does not make contact with the native lumen in which it is implanted. Preferably the outer support structures comprise of biological tissue, polymer, pure metal, alloy or a combination of the aforementioned.

The prosthesis 10 further comprises vertical struts 18. Three vertical struts 18 are shown in FIGS. 4A-4B, but any number may be used as required. In the depicted example, the struts 18 form sites of attachment 24 for the three commissures 152 of a replacement valve secured to the prosthesis 10 via sutures, adhesives, barbs or other methods known to those skilled in the art.

The vertical struts 18 can, but are not required to, collapse and expand to facilitate endovascular deployment or removal. As such, said struts 18 are elements that can but are not required to be formed using the same materials as the aforementioned frame 14. That is, they are elements that may not be capable of reversibly transitioning between a rigid and flexible mode. Similarly, it may be advantageous (in all devices of the invention) to limit the possibility of the flexible/rigid transition to occur in only a portion of the frame 14, such as just the joints 16 of the frame 14 of FIG. 4A, with the remainder of the frame formed of a material with other properties complimentary to the application of the particular device 10 (e.g. a heart valve prosthesis as depicted).

As shown in FIG. 4C, the prosthesis represents a tubular geometry in keeping with the native aortic annulus 144. Since the prosthesis 10, in its flexible state, is capable of conforming to the native geometry of the placement location, in this case the aortic annulus 144, the prosthesis 10 can be configured in vivo to the anatomy of each recipient to maintain good apposition with the surrounding tissue, in this case the wall of the aortic root 142.

The reversible nature of the encapsulated material enables repositioning and/or removal as required, both before and after full deployment. In some embodiments, long term removal (>6 weeks after deployment) may be limited by the environment of the prosthesis, e.g. if there is tissue ingrowth. As such, the prosthesis 10 may be additionally coated 20 with a material to limit tissue growth on the surface or into the lumen 22 of the prosthesis 10. Said coating can serve a second function in the context of a heart valve, by acting as a skirt to limit paravalvular leak. Selection of an additional coating material includes fluoropolymers (e.g. polytetrafluoroethylene (PTFE), expanded PTFE (ePTFE), fluorinated ethylene propylene (FEP), ethylene-tetrafluoroethylene (ETFE)), polysiloxanes, polyurethanes, polybutylenes, styrenic thermoplastic elastomers (e.g. poly(styrene-block-isobutylene-blockstyrene) (SIBS)), biological tissue or other clinically implantable material. Similarly, the prosthesis 10 may be implanted with an additional or plurality of conduits as detailed in later sections.

Referring now to FIG. 5A, in another embodiment of the present invention, the prosthesis 10 can be configured as two connected lattice rings 30a, 30b, which can be arranged in a heart valve embodiment for radial opposition at the aortic annulus 144 and the sinotubular junction 158, respectively. Between the proximal 30a and distal 30b rings are a plurality of vertical struts 18 that function to connect said rings and secure the replacement valve 12. In this embodiment, the distal ring of the prosthesis 30b has a diameter larger than the proximal ring 30a to reflect the native geometry of the aortic root 142. As shown in FIG. 5B, each tubular ring 30a, 30b has an appropriate dimension in the axial plane as to not interfere and/or obstruct the coronary ostia 154a, 154b distally or mitral valve 132 proximally.

In a modified embodiment shown in FIG. 5C, the tubular lattice rings 30a, 30b have a rhomboidal configuration whereby expansion of vertical struts 18 in the axial plane can restrict movement of the proximal and distal rings 30a, 30b within the aortic annulus 144 and sinotubular junction 158, respectively, for example. In this case, the axial force of the vertical struts 18 in the proximal and distal direction serves to resist the forward pressure of systole and backward pressure of diastole, respectively. The replacement valve 12 may be secured within the frame 14, below the coronary ostia 154a, 154b, in an intra-annular or a supra-annular position.

In yet another embodiment of the present invention, FIG. 6A illustrates a single tube configured as a helical coil 40. Axial compression or rotation in forming the coil results in a cylindrical body capable of anchoring with sufficient radial force within the surroundings, such a native aortic valve for example. Referring now to FIGS. 6B-6C, a plurality of helical 42 or vertical struts 44 may be used to reinforce geometries that conform to the native geometry the site for the prosthesis 10, such as that of the aortic root in the context of a heart valve. In that example, the contours of the prosthesis 10 can be configured to engage with aspects of the aortic root to prevent paravalvular leak and proximal or distal migration. In particular, the prosthesis 10 can engage with the annulus such that the diameter of the mid portion 11e is less than the diameter of the proximal 11a and distal 11b portions. FIG. 6D illustrates a cylindrical frame comprised of a proximal 46a and distal 46b torus fabricated from an encapsulated alloy and connected by a plurality of vertical struts 18 which may fabricated from the encapsulated alloy or from a material which does not undergo the flexible/rigid transition.

As will be readily understood by the skilled reader, a variety of dimensions for various aspects of the prosthesis 10 will allow accommodation of different sites (e.g. for different applications) and physical differences in the population. In preferred embodiments in the heart valve context, the deployed prosthesis at the level of the aortic annulus 144 is a diameter from 15 mm to 35 mm, more preferably from 19 mm to 32 mm, more preferably from 22 mm to 29 mm. The length of the prosthesis is a function of its shape and position within the aortic root. In particular, with reference to FIG. 4C, one embodiment of the heart valve prosthesis 10 extends across the native aortic valve 140 ranging 10 mm to 25 mm in length bounded proximally and distally by the mitral valve 132 and coronary ostia 154a, 154b, respectively. In contrast, as shown in FIG. 5C, another embodiment of the prosthesis 10 extends from the aortic annulus 144 to the sinotubular junction 158 ranging 30 mm to 55 mm in length. Replacement of other heart valves namely the tricuspid, pulmonic or mitral valves may require larger or smaller dimensions as determined by the native geometry of the implantation site.

The frame 14 of the prosthesis 10 can be manufactured using a number of methods. Any selected manufacturing process may favour or limit the production of certain geometries. Preferably, one aspect of the frame 14 may be manufactured using modified injection moulding techniques as described by Wiech in U.S. Pat. No. 4,197,118, hereby incorporated by reference herein. Referring now to FIG. 7A, the injection moulding apparatus 60 may include a temperature-controlled extruder 62 having a nozzle 64 through which material 66 contained in the hopper 68 is extruded into the cavity 72 of a mould 70. It may be desirable to control the temperature of the mould 70 during the injection process. With reference to FIG. 7B, the mould 70 may be formed by mating a plurality of die parts 74a, 74b, which may be made of hardened steel, aluminium and/or beryllium-copper for example.

Preferably, as shown in FIG. 7C, the mould 70 may be a solid structure comprised of a polymer or other soluble materials with sufficient mechanical rigidity and temperature stability to enable injection at the desired temperature. Said mould may be subsequently dissolved to reveal the moulded part. It may be desirable to use additive manufacturing processes to rapidly produce customised moulds. Using a variety of techniques known to those skilled in the art, the injecting system may be adapted to inject powder particles or liquids through the injection conduit and into the cavities 72 of the mould base 70. By way of example, and not limitation, powdered metals of weighted percentages: Bismuth (35.0), Indium (48.6), Tin (16.0) and Zinc (0.4) with purity of 99.99 mol percent can be mixed, cold-sintered, and homogenised whilst in their liquid state and injected into a polymeric mould as shown in FIG. 7C. Thereafter the moulded part can be revealed by post-moulding treatment with organic solvent or others methods common to those skilled in the art.

Alternatively, Patent No. US 20,120,237,385 to Wilkinson details a method of distributing metal powder particles into a mould die which may be advantageous for embodiments using mating die halves.

Another aspect of the manufacturing process relates to a method of encapsulating or coating the moulded part. This is preferably achieved by dip coating with a polymeric solution.

Therapeutic compounds may be added if appropriate. In particular, agents or combinations of agents may be added to inhibit neointimal growth, reduce inflammation and/or limit coagulation such as cyclosporine, tacrolimus, sirolimus, zotarolimus, everolimus, umirolimus, heparin, azathioprine, paclitaxel, phosphorylcholine, poly-L-lactide, mTOR inhibitors, retenoids, CDK inhibitors, HMG co-enzyme reductase inhibitors or protease inhibitors.

One method for coating the moulded part comprises: (1) fabrication of the polymeric media via dissolution of SIBS polymer pellets in solvent (10 w/v %), (2) addition of therapeutic compounds as required, (3) submersion of the prosthesis in polymeric solution, and (4) drainage and evaporation using techniques common to those skilled in the art. The process of dipping and drying is repeated to achieve a thickness that insulates surrounding biological tissue from the temperatures required to induce phase transition in the coated material. Preferably, the thickness of coating is from 0.1 mm to 3 mm, more preferably from 0.5 mm to 2.5 mm, more preferably from 1 mm to 2 mm. A number of biocompatible polymers may be utilised including those previously specified. Selection of a coating material involves consideration of properties including elasticity, durability, phase transition behaviour, heat transfer and biocompatibility in accordance with the application of a heart valve prosthesis deployed via endovascular methods.

Another method of manufacture involves multicomponent additive manufacturing. Molten alloy and softened polymer can be extruded from respective nozzles onto a manufacturing bed. The nozzles may be moved in the horizontal plane, whilst the bed may be moved vertically. Once completed, the parts may be sealed and encapsulated using a dip coating method as previously described. In another method, extruded heat shrink tubing, with one end flared, can be filled with alloy and shaped into the desired form. The narrow end of the tubing can be placed into the flared end and heated to promote the heat-shrink effect. Further encapsulation may be achieved by dip coating. In another method of manufacture blow or die cast moulding can be used to form a hollow torus. Molten alloy can then be injected at the gate position before the mould is heat sealed and encapsulated by dip coating.

Preferably, the heart valve prosthesis is advanced to a position within the native valvular annulus using retrograde endovascular methods. In some embodiments, the prosthesis in its flexible state is radially adapted to an expanded configuration via balloon inflation. In other embodiments the prosthesis in its flexible state is expanded via disengagement of a retraction sheath upon which the prosthesis returns to a predetermined configuration. Thereafter, the position and size of the prosthesis is evaluated in situ. If optimal, the prosthesis is fixed via phase transition to a rigid state. If suboptimal, the prosthesis is reversed to a smaller cross sectional area and repositioned or removed. In the long term, the prosthesis may be exchanged via re-catheterisation, phase transition to a flexible state and recapture.

In preferred embodiments, the prosthesis is delivered within a sheath of diameter between 4 mm and 10 mm, more preferably 9 mm and 5 mm, more preferably 6 mm and 8 mm. Once deployed and in its rigid state, the prosthesis has an internal diameter of between 15 mm and 35 mm, more preferably between 19 mm and 32 mm, more preferably between 22 mm and 29 mm.

Referring now to FIGS. 8A-8B, the ability to deliver the prosthesis 10 in a flexible state enables multiple mechanisms for endovascular deployment. For example, the delivery apparatus 200 can be advanced to the annulus of the native aortic valve 142 under fluoroscopic guidance such that the heart valve prosthesis 10, when deployed, will extend preferably no more than 2 mm into the left ventricular outflow tract 138.

The delivery apparatus 200 includes a nose-cone tip 212, multi-lumen catheter 214, external sheath 218 and handle 220 including the proximal handle 221 used to stabilise the delivery apparatus 200 during operation.

In one embodiment, as shown in FIG. 8A, the prosthesis 10 is deployed by inflation of a balloon 216 at the distal end of the catheter 215 using the inflation port 222 such that the prosthesis 10, in its flexible mode, is expanded to conform to the geometry of the native aortic valve 140. The use of the balloon is one way to shape the device 10 into the deployed configuration. The device 10 may have been first inserted in its rigid mode, before being caused to undergo the transition to the flexible mode (e.g. by application of a suitable stimulus) prior to shaping, or may already be in the flexible mode when it is inserted. Subsequent transition to the rigid mode permits fixation, balloon deflation and in vivo assessment. If deployment is suboptimal, it may be desirable to reposition and/or retrieve the prosthesis 10. In the flexible state, the elastic properties of the material used to encapsulate (or coat) the frame may enable recoil of the expanded prosthesis to an initial crimped configuration for repositioning and re-expansion. As such, the device 10 could be moved or removed after placement by causing the transition from rigid mode to flexible mode and then moving the device as required.

Another embodiment of the prosthesis 10, as shown in FIG. 8B, can be deployed via clockwise rotation of the actuator 230 of the delivery handle 220 to control retraction of the sheath 218 relative to the catheter 214 such that the prosthesis 10 is revealed and subsequently expands to a predetermined deployed configuration—a function of the materials and geometry of the frame. For example, the inner threads 224 of the handle 220 receive the outer threads 232 of actuator 230 such that rotation causes axial movement of the attached sheath 218 relative to the fixed catheter 214 that passes through the cylindrical recess 234. If deployment is suboptimal and the prosthesis 10 remains tethered to the catheter 214, resheathing may be achieved by advancing the sheath 218 relative to the catheter 214 via anti-clockwise rotation of actuator 230. In this manner, the prosthesis 10 may be recaptured and redeployed until such time that the delivery apparatus 200 is withdrawn from the vasculature. Alternatively, the flexible nature of the prosthesis 10 enables dynamic repositioning via movement of the catheter 214 in the axial plane to align the prosthesis 10 within the native valvular annulus 140. It may also be desirable to use wires 236 to releasably or reversibly tether the prosthesis 10 to the catheter 214. Additionally, clockwise and anti-clockwise rotation of the knob 240 may be used to position the prosthesis 10 by shortening the left and right wires, respectively. Following final positioning, the prosthesis 10 may be decoupled from the wires 236 by pressing and holding the proximal actuator 240.

As previously mentioned, removal of the prosthesis in the long term (>3 months) may be complicated by changes in the biological environment, e.g. formation of a fibrous capsule around the prosthesis and/or tissue growth through the cells of the lattice. Assuming non-degradability of the selected coating, fibrous growth will be a chronic inflammatory response dependent on various material surface properties and may lead to complete encapsulation which is ideally quiescent. With reference to FIG. 4A as one embodiment of the present invention, the prosthesis 10 may be coated to prevent tissue growth through the struts into the lumen 22 of the prosthesis 10. As previously described, ideally the properties of said coating will reduce cellular proliferation and limit the mechanical strength of the encapsulating tissue.

Referring now to FIGS. 9A-9B, in a modified embodiment, the prosthesis 10 may instead be deployed with and within a tubular conduit 300 whereby the prosthesis does not directly contact the surrounding tissue, e.g. the wall of the aortic root 142, and the conduit is made of a material that does not permit tissue growth into its interior surface. The conduit 300 can be, for example, a solid and/or continuous material without gaps that would otherwise allow tissue growth from one side to the other. FIGS. 9C-9D detail possible configurations of the prosthesis 10 within the conduit 300 whereby the conduit 300 also serves as a mechanism to secure the prosthesis 10 at the intended site, e.g. within the aortic root 142. In particular, FIG. 9C illustrates a conduit 300 configured to the native geometry with a length in the axial plane less than the prosthesis 10 such that the proximal 11a and distal 11b ends having a diameter greater than the diameter of the conduit 300, extend beyond the conduit 300. FIG. 9D illustrates a conduit 300 with cuffs at its proximal 302a and distal 302b ends, and of length greater than the prosthesis 10 in the axial plane such that the proximal 11a and distal 11b ends of the prosthesis 10 sit below and engaged with said cuffs 302a, 302b.

In another alternative embodiment, the prosthesis 10 may itself be constructed as a solid and/or continuous material without gaps that would otherwise allow tissue growth from one side to the other. The prosthesis 10 may still retain the possibility of adapting its shape by e.g. folding/crumpling/concertinaing around its perimeter.

Although the present invention has been described herein with respect to certain preferred embodiments, it is not intended to be limited. Indeed, the provision of a prosthesis with capacity to transition between flexible and rigid states under the influence of external stimuli may have significant utility to those skilled in the art beyond the disclosed embodiments to alternative embodiments or uses in other areas of the body. Insofar as applications utilise features and methods of the present invention as disclosed herein, and as claimed, such modifications are included within the scope of the present invention

### LIST OF NON-PATENT REFERENCES

Alavi S H, Groves E M, Kheradvar A. The effects of transcatheter valve crimping on pericardial leaflets. Ann Thorac Surg. 2014; 97(4):1260-6.
Bonhoeffer P, Boudjemline Y, Saliba Z, Merckx J, Aggoun Y, Bonnet D, Acar P, Le Bidois J, Sidi D, Kachaner J. Percutaneous replacement of pulmonary valve in a right-ventricle to pulmonary-artery prosthetic conduit with valve dysfunction. Lancet 2000; 356(9239):1403-1405.
Bourantas C V, van Mieghem N M, Farooq V, Soliman O I, Windecker S, Piazza N, Serruys P W. Future perspectives in transcatheter aortic valve implantation. International Journal of Cardiology 2013; 168:11-18.
Cribier A, Eltchaninoff H, Bash A, Borenstein N, Tron C, Bauer F, Derumeaux G, Anselme F, Laborde F, Leon M B. Percutaneous transcatheter implantation of an aortic valve prosthesis for calcific aortic stenosis: first human case description. Circulation 2002; 106:3006-3008.
d'Arcy J L, Prendergast B D, Chambers J B, Ray S G, Bridgewater B. Valvular heart disease: the next cardiac epidemic. Heart 2011; 97:91-93
de Buhr W, Pfeifer S, Slotta-Huspenina J, Wintermantel E, Lutter G, Goetz W A. Impairment of pericardial leaflet structure from balloon-expanded valved stents. J Thorac Cardiovasc Surg. 2012; 143(6):1417-1421.
Hannan E L, Samadashvili Z, Lahey S J, Smith C R, Culliford A T, Higgins R S, Gold J P, Jones R H. Aortic valve replacement for patients with severe aortic stenosis: risk factors and their impact on 30-month mortality. Ann Thorac Surg 2009; 87(6):1741-9.
Iung B, Baron G, Butchart E G, et al. A prospective survey of patients with valvular heart disease in Europe: The Euro Heart Survey on Valvular Heart Disease. Eur Heart J 2003; 24:1231-43.
Iung B, Cachier A, Baron G, Messika-Zeitoun D, Delahaye F, Tornos P, Gohlke-Barwolf C, Boersma E, Ravaud P, Vahanian A. Decision-making in elderly patients with severe aortic stenosis: why are so many denied surgery? Eur Heart J 2005; 26(24):2714-20.
Kouchoukos N T, Blackstone E H, Hanley F L, Kirklin J K. Kirklin/Barratt-Boyce Cardiac surgery: Morphology, Diagnostic Criteria, Natural History, Techniques, Results, and Indications. Elsevier/Saunders 2013, Philadelphia.
Stortecky S, Buellesfeld L, Wenaweser P, Windecker S. Transcatheter aortic valve implantation: prevention and management of complications. Heart 2012; 98(S4):iv52-iv64.
Walther T, Blumenstein J, van Linden A, Kempfert J. Contemporary management of aortic stenosis: surgical aortic valve replacement remains the gold standard. Heart 2012; 98: Suppl 4:iv23-iv29.

1. An implant device for insertion in a human or animal body, the device comprising:
a frame; and
wherein at least a portion of the frame is capable of reversibly transitioning between a solid state rigid mode and a liquid state flexible mode, thereby allowing the device to change its shape.

2. The device according to claim 1, wherein the device is or includes a prosthesis such as a heart valve, a stent, an otorhinolaryngology implant, a bariatric sleeve, a urinary sphincter or an inferior vena cava filter, or a catheter, surgical retractor or endoscopic speculum.

3. The device according to claim 1, wherein the frame further comprises elements which are not capable of reversibly transitioning between a rigid and flexible mode.

4. The device according to claim 1, wherein at least said portion of the frame comprises a core surrounded by an encapsulating layer.

5. The device according to claim 4, wherein the encapsulating layer comprises biological or polymeric material.

6. The device according to claim 4, wherein said portion is capable of reversibly transitioning between a rigid and flexible mode due to a change in the properties of the core.

7. The device according to claim 4, wherein said portion of the frame is configured to undergo the transition in mode under the influence of an external stimulus.

8. The device according to claim 7, wherein the material properties of the core are such that the core undergoes a phase transition at a temperature of from 35° C. to 80° C., more preferably from 40° C. to 70° C., and even more preferably from 45° C. to 60° C.

9. The device according to claim 4, where the encapsulation layer comprises fluoropolymers, in particular polytetrafluoroethylene (PTFE) or expanded PTFE (ePTFE) or fluorinated ethylene propylene (FEP) or ethylene-tetrafluoroethylene (ETFE), polysiloxanes, polyurethanes, polybutylenes, and/or styrenic thermoplastic elastomers, in particular poly(styrene-block-isobutylene-blockstyrene) (SIBS).

10. The device according to claim 1, wherein said portion or said core material comprises a eutectic alloy, ethylene butyl acrylate copolymer, ethylene methyl acrylate copolymer, ethylene acrylic acid copolymer, ethylene methacrylic acid copolymer, polybutylene, poly(epsilon-caprolactone), thermoplastic polyolefin elastomer/plastomer, thermoplastic polyurethane elastomer, polyamide, polylactic acid, poly(n-isopropylacrylamide) and/or cellulose acetate butyrate.

11. The device according to claim 1, wherein said portion, or said core if the portion comprises an encapsulating layer, has a Young's modulus of at least 0.1 GPa in the rigid mode, preferably at least 1 GPa.

12. The device according to claim 1, wherein said portion, or said core if the portion comprises an encapsulating layer, has a Young's modulus of less than 0.1 GPa in the flexible mode, preferably less than 0.01 GPa.

13. A method of using the device of claim 1, the method comprising:
causing the device to undergo transition from one of the rigid or flexible modes to the other.

14. A method of inserting the device of claim 1 into a human or animal body, the method comprising:
causing the device to undergo transition from the rigid mode to the flexible mode;
shaping the device into a deployed configuration; and
causing the device to undergo transition from the flexible mode to the rigid mode.

15. The method according to claim 14, further comprising inserting the device into the human or animal body either before or after the step of causing the device to undergo transition from the rigid mode to the flexible mode.

16. A method of moving, or removing from a human or animal body, the device of claim 1, the method comprising:
causing the device to undergo transition from the rigid mode to the flexible mode; and
moving the device, optionally including removing the device from the human or animal body.

17. A method according to claim 13, wherein the step of causing the device to undergo transition from the rigid mode to the flexible mode comprises heating said portion of the device.

18. A method according to claim 17, wherein the source of heat is external to the body, internal to the body but separated from the frame, or in direct contact with the frame.
